(19) United States
(12) Patent Application Publication
(10) Pub. No.: US 2020/0323180 A1
(43) Pub. Date: Oct. 15, 2020

(54) METHODS FOR THE PRODUCTION OF STERILE FISH AND OTHER EGG-PRODUCING AQUATIC ANIMALS AND COMPOUNDS FOR USE IN THE METHODS

(71) Applicant: ACD PHARMACEUTICALS AS, Leknes (NO)

(72) Inventor: Juan José JIMÉNEZ MARTÍNEZ, Moss (NO)

(73) Assignee: ACD PHARMACEUTICALS AS, Leknes (NO)

(21) Appl. No.: 16/771,940

(22) PCT Filed: Dec. 13, 2018
(86) PCT No.: PCT/NO2018/050308

§ 371 Date: Jun. 11, 2020

(30) Foreign Application Priority Data
Dec. 15, 2017 (NO) 20172005

(51) Int. Cl. A01K 67/027; C12N 15/113

(52) U.S. Cl. CPC A01K 67/027; C12N 15/113; C12N 2310/315; A01K 2267/02; C12N 2310/341; A01K 2227/40; A01K 2207/05; C12N 2310/3231

(57) ABSTRACT

This invention relates to methods and compounds for the production of sterile fish and other egg-producing aquatic animals. The methods include/the compounds are useful to cause disruption of gonadal development in fish or other egg-producing aquatic animals through the administration of compounds that lead to the failure of fertile gonadal development, and thus to reproductively sterile fish or other egg-producing aquatic animals. The methods and compounds are for use in e.g. aquaculture, the aquarium trade or control of invasive species.

### RELATED APPLICATIONS

This application is a US National Stage Entry under U.S.C. § 371 of International Patent Application PCT/NO2018/050308, filed Dec. 13, 2018, which claims priority to Norwegian Patent Application NO 20172005, filed Dec. 15, 2017, all of which are hereby incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Dec. 13, 2018, is named ACDPA1701PCTseqlist.txt and is 1 KB in size.

This invention relates to methods and compounds for the production of sterile fish and other egg-producing aquatic animals. The methods include/the compounds are useful to cause disruption of gonadal development in fish or other egg-producing aquatic animals through the administration of compounds that lead to the failure of fertile gonadal development, and thus to reproductively sterile fish or other egg-producing aquatic animals. The methods and compounds are for use in e.g. aquaculture, the aquarium trade or control of invasive species.

Aquaculture is currently one of the fastest growing food-producing industries and is becoming increasingly important to resolve the current and projected global shortfalls in aquatic foods and seafood availability. Optimization of methods and procedures used in aquaculture is increasingly necessary to maximize animal welfare/health and food production and to minimize ecological impact, thereby achieving long-term environmental sustainability of our seafood supplies.

Sterilization, i.e. induced infertility of farmed fish and other egg-producing aquatic animals enhances their growth rate by increasing the conversion of food energy to muscle growth, instead of gonadal development. In addition, if escaped from aquaculture operations to the environment, reproductively sterile farmed fish and other egg-producing aquatic animals, including domesticated, non-native or genetically modified species, will not be able to reproduce or inter-breed with wild populations. This will assist biological containment and prevent genetic contamination of wild populations and/or the establishment in the wild of domestic, non-native or genetically modified farmed fish and other egg-producing aquatic animals.

Sterility in fish and other egg-producing aquatic animals can be achieved by disrupting gonadotropin releasing hormone (GnRH) cell development and/or primordial germ cell (PGC) development, migration and colonization in the gonad of the embryo. This results in failure of gonad development and/or failure of full and proper gonadal functioning, and ultimately the generation of reproductively sterile fish and other egg-producing aquatic animals.

Compounds known to disrupt GnRH cell and/or PGC development, migration and/or survival include antisense oligonucleotides (AON), which inactivate mRNAs essential for GnRH cell and/or PGC formation, migration and/or survival by blocking their translation or otherwise interfere with their processing. Z. Linhartová et al., Theriogenology 84 (2015), 1246-1255 disclose the sterilization of sturgeon by microinjection into sturgeon embryos of antisense morpholino oligonucleotides, which knock-down dead end (dnd) gene expression. Dnd is a vertebrate-specific gene encoding a RNA-binding protein, which is crucial for migration and survival of PGCs.

T. Wong and Y. Zohar, Sci. Rep. 5 (2015), 15822 disclose a bath-immersion method to produce infertile zebrafish which includes the use of Vivo-conjugated morpholino oligonucleotides against zebrafish dnd.

WO 2015/073819 A1 and WO 2016/187198 disclose methods for the production of reproductively sterile fish and egg-producing aquatic animals for e.g. aquaculture. Compounds for rendering such animals sterile may be delivered to the eggs of such animals prior to fertilization or water activation of said eggs or post fertilization and water activation by contacting the eggs in an immersion medium including the compounds of interest, and optionally a molecular transporter.

Generally, in the antisense technology, mRNA is targeted by Watson-Crick hybridization of a complementary AON. The goal of inhibiting gene expression in a specific way may be accomplished by preventing mRNA maturation, blocking translation or by induction of degradation. To be effective the AON must be able to enter the cell, be stable toward nucleases, be non-toxic and show high binding affinity and specificity toward the target mRNA.

Considerable progress with respect to stability and binding has been made by use of chemically modified AONs. An example of such chemically modified AONs oligonucleotides with a phosphorothioate backbone instead of a phosphodiester backbone or a backbone comprising methylenemorpholine rings (i.e. morpholino oligonucleotides), which enhances resistance to enzymatic degradation. Another example is locked nucleic acids (LNA), a nucleoside comprising a bicyclic sugar moiety comprising a 4′-CH2-O-2′ bridge, which “locks” the sugar ring in a conformation that exhibits increased binding affinity towards complementary RNA.

This invention relates to methods and compounds for the production of sterile fish and other egg-producing aquatic animals by disruption of gonadal development in fish or other egg-producing aquatic animals through the administration of gapmers.

In a first embodiment, the invention provides a method for producing sterile egg-producing aquatic animals, said method comprises transfecting such eggs with a gapmer that is effective to render individuals produced therefrom sterile, i.e. a gapmer that suppresses expression of a protein that is essential for embryonic germ cell development by promoting degradation of gapmer-targeted mRNA, thereby causing failure of fertile gonadal development.

In a second embodiment, the invention provides a gapmer for use in a method for producing sterile egg-producing aquatic animals, wherein said method comprises transfecting such eggs with the gapmer, which is effective to render individuals produced therefrom sterile.

In a third embodiment, the invention provides a gapmer that suppresses expression of a protein that is essential for embryonic germ cell development in egg-producing aquatic animals.

In a forth embodiment, the invention provides a composition comprising a gapmer that suppresses expression of a protein that is essential for embryonic germ cell development in egg-producing aquatic animals.

In a fifth embodiment, the invention provides a composition comprising a gapmer that suppresses expression of a protein that is essential for embryonic germ cell development in egg-producing aquatic animals for in a method for producing sterile egg-producing aquatic animals, wherein said method comprises transfecting such eggs with the composition.

As used herein, the term egg-producing aquatic animals refers to fish and such other aquatic animals which lay eggs in water, including crustaceans and/or molluscs. In a preferred embodiment, the invention related to compositions and methods for producing sterile fish.

Accordingly, egg-producing aquatic animals includes all fish species, including, but not limited to salmonids, e.g. Atlantic salmon, coho salmon, chinook salmon, chum salmon, sockeye salmon, pink salmon, masu salmon, trout species such as rainbow trout, brook trout, brown trout, common grayling, Arctic grayling, Arctic char, moronids, e.g. bass, striped bass, white bass, striped-white bass hybrids, yellow bass, perch such as white perch, yellow perch or European perch, bass-perch hybrids, cichlids, e.g. Nile tilapia, blue tilapia, blue-Nile tilapia hybrids, Mozambique tilapia, cyprinids, e.g. zebrafish and other carp species, breams, e.g. seabreams, porgies, gadids, e.g. cod, haddock, whiting, pollock and catfish species.

Further, egg-producing aquatic animals include all other aquatic animals which lay egg in water, including but not limited to crustaceans such as shrimp, prawn, lobster, crayfish and crabs and mollusca species, including but not limited to oysters, mussels, clams such as scallops or geoducks, squid, octopus and cuttlefish.

In one preferred embodiment, the egg-producing aquatic animal is one for aquaculture such as a salmon, a trout, a bass, a tilapia, a carp, a catfish, a shrimp, a prawn, an oyster, a geoduck or an abalone. In another preferred embodiment, the egg-producing aquatic animal is one for aquarium trade, e.g. a zebrafish, a goldfish, a catfish, a tetra, a hatchet fish, a pencil fish, a goby, a damselfish, a clownfish, or chromis.

In another preferred embodiment, the egg-producing aquatic animal is a fish,

e.g. a salmonid, moronid, cichlid, gadid, pangasid, ictalurid or cyprinid such as a salmon, a trout, a bass, a tilapia, a carp or a catfish.

As used herein, the term “gapmer” refers to a single-stranded, antisense oligonucleotide having a contiguous wing-gap-wing sequence. The gap comprises a continuous stretch of RNase H recruiting nucleotides. The gap is flanked by a 5′ wing region and a 3′ wing region, which each comprise affinity-enhancing chemically modified nucleotides.

The gap of the gapmer may consist of 6 to 20 linked RNase H recruiting nucleotides, such as 6 to 20, 6 to 18, 6 to 16, 6 to 14, 6 to 12 or 6 to 10 or 7 to 20, 7 to 18, 7 to 16, 7 to 14, 7 to 12, 7 to 10 or 8 to 20, 8 to 18, 8 to 16, 8 to 14, 8 to 12, 8 to 10 or 9 to 20, 9 to 18, 9 to 16, 9 to 14, 9 to 12 or 9 to 10 or 10 to 20, 10 to 18, 10 to 16, 10 to 14 or 10 to 12.

Each nucleotide of the gap may be a 2′-deoxynucleotide. In certain embodiments, the gap comprises one or more modified nucleotides that are “DNA-like”, i.e. they have similar characteristics to DNA, such that a duplex comprising the gapmer and the target RNA molecule is capable of recruiting and activating RNase H. Examples of such modified DNA-like nucleotides are 2′-deoxynucleotides with a phosphorothioate group rather than a phosphodiester group. In a certain embodiment, each nucleotide of the gap is a 2′-deoxynucleotide comprising a phosphorothioate group. Other examples of modified nucleotides that are DNA-like are nucleotides comprising 2-fluoroarabinose (FANA), α-L-LNA or C4′-substituted nucleotides, such as C4′-methyl-DNA nucleotides, e.g. C4′-hydroxymethyl-DNA nucleotides, C4′mercapto-methyl-DNA nucleotides or C4′-aminomethyl-DNA nucleotides. In certain embodiments, one or more nucleotides of the gap is not a 2′-deoxynucleoside and is not DNA-like nucleotide. In certain such embodiments, the gapmer nonetheless supports RNase H activation, e.g., by virtue of the number or placement of the non-DNA or non-DNA-like nucleotides.

The 5′ wing region of the gapmer may consist of 1 to 8 linked nucleotides, such as 1 nucleotide, 2 nucleotides, 3 nucleotides, 4 nucleotides, 5 nucleotides, 6 nucleotides, 7 nucleotides or 8 nucleotides.

The 3′ wing region of the gapmer may consist of 1 to 8 linked nucleotides, such as 1 nucleotide, 2 nucleotides, 3 nucleotides, 4 nucleotides, 5 nucleotides, 6 nucleotides, 7 nucleotides or 8 nucleotides.

The 5′ wing region may consist of the same number of linked nucleotides as the 3′ wing region or the 5′ wing region and the 3′ wing region may consist of a different number of linked nucleotides.

Each nucleotide of the wing region may be an affinity-enhancing chemically modified nucleotide. Examples of such nucleotides are LNA, e.g. LNA in the α-L-conformation or β-D-conformation, 2′-alkylated RNA nucleotides, e.g. 2′-O-methylRNA nucleotides or 2′-O-methoxyethyl-RNA nucleotides.

The gapmer may consist of 8 to 36 nucleotides. In some embodiments, the gapmer consists of 10 to 22 nucleotides, such as 12 to 18, 13 to 17 or 12 to 16 nucleotides, e.g. 12, 13, 14, 15 or 16 nucleotides or 14, 15, 16, 17, 18, 19 or 20 nucleotides. In some embodiments, the gapmer consists of no more than 22

nucleotides, e.g. no more than 20 nucleotides or no more than 18 nucleotides, such as 15, 16 or 17 nucleotides. In some embodiments, the gapmer comprises less than 20 nucleotides.

The wing-gap-wing motif of the gapmer may be 5-8-5, 5-6-5, 4-10-4, 4-8-4, 46-4, 3-12-3, 3-10-3, 3-8-3, 2-16-2, 2-14-2, 2-12-2, 2-10-2, 1-16-1, 1-14-1, 1-12-1, 110-1, 2-8-2, 1-8-1, 3-6-3 or 1-6-1.

The internucleoside linkages between the nucleotides in the gapmer may be all phosphorothioate internucleoside linkages. In some such embodiments, the phosphorothioate internucleoside linkages are stereodefined, i.e. [all Rp] configuration or [all Sp] configuration. In other embodiments, the phosphorothioate internucleoside linkages are not stereodefined, i.e. racemic mixtures of Rp and Sp. In yet another embodiment, the phosphothioate internucleoside linkages within the 5′ wing region and/or the gap and/or the 3′ wing region are stereodefined.

Gapmers can be synthesized by methods known in the art. They are commercially available, e.g. from Qiagen, Bio-Synthesis Inc., Exiqon and the like.

The sequence of the gapmer can be designed using available gene sequence database and bioinformatics tools to achieve high target affinity, sequence specificity and biological stability. Companies offering custom gapmer synthesis often also offer tools for their design.

As used herein, the term “sterile” egg-producing aquatic animals refers to an individual that is unable to sexually reproduce. Sterile egg-producing aquatic animals are defined as individuals that are unable to reach sexual maturity or to reproduce when reaching the age of sexual maturity.

The production of sterile egg-producing aquatic animals according to the invention involves the disruption of gonadal development in the embryo developed from the eggs of such animals after fertilization. This is achieved by transfecting the eggs with a gapmer that is capable of the disruption of gonadotropin releasing hormone (GnRH) cell development and/or primordial germ cell (PGC) development by knockdown of mRNA and thus disrupting the generation of a gene product responsible for or involved in GnRH cell and/or PGC development, migration and/or colonization.

The gapmer used in the method of the invention/according to the invention catalyzes RNase H-dependent degradation of complementary mRNA targets, thus “knocking down” such targets: the wing regions of the gapmer achieve sufficient target binding affinity and confer nuclease resistance. When the gapmer is hybridized to its target RNA, the central gap activates RNase H cleavage of the opposing RNA strand and thus the degradation of the target RNA. The gapmer for use in the method of the invention/according to the invention thus has a sequence which is suitable to target mRNA of a gene which is essential for embryonic germ cell development, cleave said RNA and suppress the expression of a protein that is essential for embryonic germ cell development, thereby causing failure of fertile gonadal development.

The gapmer for use in the method of the invention/according to the invention has a sequence suitable for targeting RNA of genes that are essential for embryonic germ cell development. The specific sequence of the gapmer is dependent on the target RNA, the location of binding within the target RNA and the species of the egg-producing aquatic animal. In a preferred embodiment, the gapmer for use in the method of the invention/according to the invention has a sequence suitable for targeting dead end (dnd), nanos, vasa, piwi, gnrh or fsh receptor RNA and more preferably a sequence suitable for targeting dnd RNA. Knockdown of said genes results in the failure of fertile gonadal development and ultimately generates sterile egg-producing aquatic animals.

In one embodiment, the invention provides a gapmer having a sequence suitable for targeting dead end (dnd), nanos, vasa, piwi, gnrh or fsh receptor RNA. In a preferred embodiment, the invention provides a gapmer having a sequence suitable for targeting dnd RNA.

The term “targeting” as used herein refers to the gapmer binding to/hybridizing to a complementary RNA.

The term “egg” as used herein refers to an egg cell (ovum), i.e. an unfertilized egg or the vessel containing a zygote or an embryo, i.e. a fertilized egg.

In one embodiment, the eggs transfected by the method of the invention/with the gapmer of the invention are unfertilized eggs or pre-water activated fertilized eggs. In such eggs, the chorion, i.e. the extracellular protective outer envelope surrounding the egg, shows numerous pore channels which allow for the passage of molecules through the chorion into the egg. In another embodiment, the eggs are fertilized eggs. In such eggs, the structure of the chorion has changed such that the passage of molecules is limited.

In one embodiment, the transfection of the eggs may be carried out by microinjection of the gapmer into said eggs, e.g. by using a commercially available microinjector. Typically, the gapmer is dissolved in water or a buffer and injected at a concentration of 5-5000 ng/μl, e.g. 50-500 ng/μl or 100-400 ng/μl. The eggs are preferably fertilized eggs at the one-cell stage.

In another embodiment, the eggs are transfected by immersion in an aqueous immersion medium. Generally, the immersion medium comprises at least the gapmer and water. In one embodiment, the aqueous immersion medium consists of the gapmer and water. In another embodiment, the immersion medium may further comprise other compounds, including compounds that assist or promote the transfection and/or compounds that are beneficial to the embryos/egg-producing aquatic animals hatched from the transfected eggs.

Compounds which assist or promote the transfection include salts such as acetates, citrates, borates, phosphates and the like, buffering agents which keep the pH of the aqueous immersion medium within a desired range, preferably in the range of 7-9, including Tris, HEPES, MOPS and the like and chelating agents such as EDTA, amino acids like glutamine, glycine and the like.

Compounds that are beneficial to the embryos/egg-producing aquatic animals hatched from the transfected eggs include hormones, growth promoters, protective antigens, antibiotics, nutrients and the like.

Other compounds which may be present in the aqueous immersion medium include culture medium for eggs of egg-producing aquatic animals, ovarian fluid, serum, protease inhibitors and the like.

In one embodiment, the eggs are immersed in the aqueous immersion medium and the gapmer is naturally taken up by the eggs. Such unassisted uptake is also known as gymnosis. The immersed eggs may be briefly sonicated. Gapmer concentrations may be in the range of 1 nM to 250 μM, e.g. 1 nM to 5 nM or 5 nM to 50 nM or 10 nM to 20 nM or 100 nM to 250 μM, e.g. 100 to 250 nM, 250 to 500 nM, 500 to 1000 nM, 1 μM to 50 μM, 50 μM to 100 μM, 100 μM to 150 μM, 150 μM to 200 μM or 200 μM to 250 μM, such as at least 5 nM, at least 10 nM, at least 20 nM, at least 50 nM, at least 100 nM, at least 250 nM, at least 500 nM, at least 1 μM, at least 50 μM, at least 100 μm, at least 150 μM, at least 200 μM.

In another embodiment, the eggs are immersed in the aqueous immersion medium and transfected by electroporation, using a commercially available electroporation system. Typical conditions include 1 to 32 pulses and field strengths in the range of 10 to 150 V/cm. In electroporation, an electrical field is applied to the target cell in order to increase the permeability of the cell membrane, allowing compounds to be introduced into the cell. Hence, this embodiment is suitable for unfertilized eggs, pre-water activated fertilized eggs and for fertilized eggs.

In yet another embodiment, eggs are immersed in an aqueous immersion medium comprising a transfection agent. The immersed eggs may be briefly sonicated. Suitable transfection agents are cationic agents such as cationic lipids/liposomes, cationic polymers or dendrimers such as histones, spermine, DEAE-dextran, polylysine or polyarginine or cationic peptides such as arginine and/or lysine-rich peptides, which form a complex based on electrostatic interaction with the negatively charged gapmer. Such transfection agents are commercially available, e.g. Lipofectamine®, a liposome formulation of the polycationic lipid 2,3-dioleyloxy-N-[2(sperminecarboxamido)ethyl]-N,N-dimethyl-1-propanaminium trifluoroacetate (DOSPA) and the neutral lipid dioleoyl phosphatidylethanolamine (DOPE) available from e.g. Thermofisher, Lipofectamine® 2000, available from Thermofisher.

Oligofectamine™ or Lipofectin® a 1:1 (w/w) liposome formulation of the cationic lipid N-[1-(2,3-dioleyloxy)propyl]-n,n,n-trimethylammonium chloride (DOTMA) and (DOPE), also available from Thermofisher.

The concentration of the gapmer in the aqueous immersion medium depends on the type of egg (the animal species) and is typically about 0.1 nM to 100 μM. The transfection of fertilized eggs may require a higher concentration of the gapmer in the aqueous immersion medium compared to the transfection of unfertilized eggs or prewater activated fertilized eggs. For the transfection of fertilized eggs, the concentration of the gapmer in the aqueous immersion medium may be about 1 nM to 100 μM, e.g. 1 nM to 100 nM, 100 nM to 1 μM, 1 μM to 20 μM, 20 μM to 80 μM, 20 μM to 60 μM or 20 μM to 40 μM, such as at least 1 nM, at least 10 nM, at least 50 nM, at least 100 nM, at least 1 μM, at least 10 μM, at least 20 μM, at least 40 μM, at least 60 μM or at least 80 μM.

For the transfection of unfertilized eggs or pre-water activated fertilized eggs, the concentration of the gapmer in the aqueous immersion medium may be about 0.1 nm to 1 nM, 1 nM to 10 nM, 10 nM to 100 nM, 1 μM to 40 μM, e.g. 1 μM to 30 μM, 1 μM to 20 μM, 1 μM to 15 μM, 1 μM to 10 μM or 1 μM to 5 μM, such as at least 0.1 nM, at least 1 nM, at least 10 nM, at least 100 nM, at least 1 μM, at least 3 μM, at least 5 μM, at least 10 μM, at least 15 μM, at least 20 μM or at least 30 μM. The amount of transfection agent is typically adapted to the concentration of gapmer and chosen based on the instructions provided by the transfection agent supplier.

The time required for satisfactory transfection, i.e. time required for the eggs to be immersed in the immersion medium will depend on the method of transfection and on the type of egg (animal species).

If transfection is carried out by gymnosis, the eggs may be immersed in the aqueous immersion medium comprising the gapmer for 1 to 144 hours, e.g. 1 hour to 2 hours, 2 hours to 6 hours, 6 hours to 12 hours, 12 hours to 24 hours, 24 hours to 36 hours, 36 hours to 48 hours, 48 hours to 120 hours or 4 hours to 12 hours, 24 hours to 96 hours, or 36 to 72 hours, such as at least 2 hours, at least 4 hours, at least 6 hours, at least 8 hours, at least 12 hours, at least 24 hours, at least 36 hours, at least 48 hours, at least 60 hours, at least 72 hours, at least 84 hours, at least 96 hours, at least 108 hours, at least 120 hours or at least 132 hours. For long immersion periods, it may be beneficial to replace the immersion medium with fresh immersion medium.

If transfection is carried out in an immersion medium comprising a transfection agent, typically, the eggs may be immersed in the immersion medium comprising the gapmer and the transfection agent for 1 to 96 hours, e.g. 1 to 2 hours, 2 to 4 hours, 4 to 8 hours, 8 to 10 hours, 10 to 12 hours, 12 to 24 hours, 24 to 48 hours, 48 to 72 hours or 72 to 96 hours.

If transfection is carried out by electroporation, the eggs may be immersed in the aqueous immersion medium comprising the gapmer for a very short time, typically milliseconds to seconds, depending on the number of pulses and time interval between each pulse.

The temperature of the aqueous immersion medium depends on the type of egg/animal species and the duration of the transfection, i.e. the time the eggs have to be kept in the aqueous immersion medium for them to be transfected. For gymnosis and for transfection with the help of a transfection agent, the temperature of the aqueous immersion medium will typically be kept in the range of the water temperature of the eggs' natural habitat. For eggs of cold water fish such as salmon, the temperature of the immersion medium is typically kept to 2 to 8° C., e.g. 2 to 4° C., 4 to 6° C. or 3 to 8° C., while for eggs of warm water fish such as tilapia, the temperature of the immersion medium is typically kept to 21 to 25° C.

The success of the transfection, i.e. the degradation of the target RNA and suppression of protein expression, can easily be determined by quantitative PCR (qPCR); hence, successfully transfected eggs can be identified and selected and there is no need to follow the embryos until maturation of the gonads to show that GnRH cell and/or PGC development, migration and/or colonization was disrupted and resulting adult egg-producing aquatic animals will be reproductively sterile.

After transfection of unfertilized eggs, such eggs are fertilized, i.e. contacted with male sperm of the egg-producing aquatic animal. Prior to fertilization, the eggs may be washed and/or immersed in a medium with is suitable for fertilization before being transferred into a habitat/an environment, which allows the embryo to develop, grow and ultimately the egg-producing aquatic animal to hatch. After transfection of pre-water activated fertilized eggs, such eggs are water-activated. Prior to water activation the eggs may be washed with water or a suitable medium before being transferred into a habitat/an environment, which allows the embryo to develop, grow and ultimately the egg-producing aquatic animal to hatch. After transfection of fertilized eggs, such eggs may be washed with water or a suitable medium before being transferred into a habitat/an environment, which allows the embryo to develop, grow and ultimately the egg-producing aquatic animal to hatch.

The method of the invention is useful to produce reproductively sterile aquatic egg-producing animals. Sterilization (induced infertility) of farmed aquatic egg-producing animals, e.g. farmed fish, enhances their growth rate by increasing the conversion of food energy to muscle growth, instead of gonadal development. In addition, if escaped from aquaculture operations to the environment, sterile egg-producing aquatic animals, including domesticated, non-native or genetically modified species, will not be able to reproduce or inter-breed with wild stock. This will assist biological containment and prevent genetic contamination of wild populations and/or the establishment in the wild of domestic, non-native or genetically modified farmed aquatic egg-producing animals.

In another embodiment, the invention provides a gapmer which is effective to transfect eggs from egg-producing aquatic animals and render individuals produced therefrom sterile, i.e. a gapmer that suppresses expression of a protein that is essential for embryonic germ cell development in egg-producing aquatic animals. The gapmer may be a single sequence or may be a mixture of two or more sequences, wherein each of them suppresses expression of a protein that is essential for embryonic germ cell development in egg-producing aquatic animals.

The gapmer of the invention has preferably a sequence suitable for targeting dead end (dnd), nanos, vasa, piwi, gnrh or fsh receptor RNA and more preferably a sequence suitable for targeting dnd RNA in egg-producing aquatic animals. In a preferred embodiment, the gapmer of the invention has a sequence suitable for targeting dead end (dnd), nanos, vasa, piwi, gnrh or fsh receptor RNA and more preferably a sequence suitable for targeting dnd RNA in fish, preferably in salmonids, moronids, cichlids, pangasids, ictalurids or cyprinids. In another preferred embodiment, the gapmer of the invention has a sequence suitable for targeting dnd RNA in salmonids, such as Atlantic salmon, coho salmon, chinook salmon, chum salmon, sockeye salmon, pink salmon, masu salmon, trout species such as rainbow trout, brook trout, brown trout, common grayling, Arctic grayling and Arctic char.

In one embodiment, the gapmer of the invention has a sequence suitable for targeting dnd RNA in Atlantic salmon, e.g. SED ID NO: 1, SED ID NO: 2 or SED ID NO: 3 or a variant thereof. In another embodiment, the gapmer of the invention consists of a gapmer of SED ID NO: 1 and a gapmer of SED ID NO: 2 (or variants thereof) or a gapmer of SED ID NO: 1 and a gapmer of SED ID NO: 3 (or variants thereof) or a gapmer of SED ID NO: 2 and a gapmer SED ID NO: 3 (or variants thereof) or a gapmer of SED ID NO: 1, a gapmer of SED ID NO: 2 and a gapmer of SED ID NO: 3 (or variants thereof).

The term “variant” denotes gapmers of the same sequence comprising other chemical modifications and gapmers that cover the whole or partial sequences listed above which are suitable for targeting dnd RNA in Atlantic salmon. Also included are variants of these gapmers having 80%, 85%, 90%, 95%, 97%, 98%, or 99% (including all integers in between) sequence identity or sequence homology to any one of SEQ ID NOS: 1, 2 and 3, and/or variants that differ from these sequences by about 1, 2, 3, 4 or 5 nucleotides which are suitable for targeting dnd RNA in Atlantic salmon. In embodiments, the variants disrupt dead end mRNA thereby ensuring loss of function of said mRNA in Atlantic salmon.

In another embodiment, the invention provides a composition comprising a gapmer that suppresses expression of a protein that is essential for embryonic germ cell development in egg-producing aquatic animals and a pharmaceutically acceptable carrier.

The term “pharmaceutically acceptable” denotes a carrier that is suitable for use in a veterinary pharmaceutical product and which fulfills the requirements related to, e.g.

safety, bioavailability and tolerability. Suitable carriers are water and aqueous buffers.

The composition may further comprise pharmaceutically acceptable excipients such as salts, stabilizers, pH-regulating agents and the like.

In one embodiment, the composition is the aqueous immersion medium mentioned earlier in this application.

The invention will be illustrated by the following non-limiting examples.

### EXAMPLES

### Example 1

### Transfection of Fertilized Salmo Salar Eggs by Immersion in an Aqueous Immersion Medium Comprising a Gapmer that Targets Dead End mRNA

Gapmers 1-3 that target Salmo salar dead end (dnd) mRNA and gapmer 4 having a non-dnd specific sequence (negative control) were designed using the online Qiagen gapmer design tool and purchased from Qiagen:

Gapmer 1:
(SEQ ID NO: 1)
TTGAACGCTCCTCCAT
Gapmer 2:
(SEQ ID NO: 2)
GGAGCAGCGAGGAGGT
Gapmer 3:
(SEQ ID NO: 3)
GCAAAACATTTAAGTA
Gapmer 4:
(SEQ ID NO: 4)
GCTCCCTTCAATCCAA

The gap of the gapmers comprised phosphorothioate-modified nucleotides while the wings comprised LNAs.

Salmo salar eggs were fertilized in an aqueous immersion medium (150 mM NaCl, 3.3 mM KCl, 20 mM Tris-HCl, pH 8.4) for 10 minutes before the fertilized eggs were collected and rinsed once in the aqueous immersion medium. The eggs were placed into 10 test tubes (3 eggs/test tube) and immersed in an aqueous immersion medium (150 mM NaCl, 3.3 mM KCl, 20 mM Tris-HCl, pH 8.4) containing either 10 nM of gapmer 1, 2, 3 or 4 (2 test tubes each) or no gapmer (2 test tubes). Of the two equal test tubes, one was briefly sonicated (10 s, 42 kHz). All test tubes were stored at 8° C. in the dark for 4 hours, then RNA was extracted according to the TRIzol method, and treated with DNase to completely remove any DNA from the extracted RNA.

RNAs were reverse transcribed into cDNAs using the Qiagen OneStep RT-PCR kit, following the manufacturer's instructions. PCR was carried out with a BioRad MyCycler thermocycler using the OneTaq Polymerase kit (New England Biolabs) and following the manufacturer's instructions. The following dnd-specific primers were used (amplicon size 401 bp):

Salgap2-F:
(SEQ ID NO: 5)
GAGCGTTCAAGTCAGGTGTTG
Salgap2-R:
(SEQ ID NO: 6)
CAGAGCTGACGTTTCTCCGT

PCR amplification of a common Salmo salar house-keeping gene was carried out to control integrity of the extracted RNA/transcribed cDNA. All samples showed an equally strong band for the house-keeping gene. No-template PCR controls were blank, i.e. showing that there was no dnd template contamination in the PCR reagents and/or the equipment which could produce false results.

Eggs which had been immersed in immersion medium only (without gapmer) gave strong PCR signals, showing that the dnd-mRNA was detectable in such eggs. Eggs which had been immersed in immersion medium comprising gapmer 4 also gave strong PCR signals, showing that the random gapmer sequence did not target dnd mRNA.

Eggs which had been immersed in immersion medium comprising gapmer 1 gave very weak (with sonication) and no PCR signal (without sonication), showing that the eggs had been successfully transfected and that gapmer 1 was effective to target and destroy dnd-mRNA, i.e. knockdown of the dead end gene which is crucial for the migration and survival of PGCs. If the eggs had been allowed to develop, the fish hatched therefrom would have been reproductively sterile.

Eggs which had been immersed in immersion medium comprising gapmer 2 gave a weak PCR signal (with or without sonication), showing that the eggs had been successfully transfected and that gapmer 2 was to a large degree effective to target and destroy dnd-mRNA. It is likely that gapmer 2 could show equally good effectivity under optimized incubation conditions, e.g. longer incubation time.

Eggs which had been immersed in immersion medium comprising gapmer 3 gave a strong PCR signal (with sonication) and no PCR signal (without sonication). It is believed that the strong PCR signal in the sonicated eggs is not due to failure to transfect the eggs or lack of effectiveness of the gapmer but rather due to the eggs having suffered from the mechanical treatment. This hypothesis is supported by the absence of the PCR signal in the non-sonicated eggs which shows that the eggs had been successfully transfected and that gapmer 3 was effective to target and destroy dnd-mRNA, i.e. knockdown of the dead end gene which is crucial for the migration and survival of PGCs. If the eggs had been allowed to develop, the fish hatched therefrom would have been reproductively sterile.

### Example 2

### Transfection of Fertilized Salmo Salar Eggs by Microinjection of a Gapmer that Targets Dead End mRNA

Salmo salar eggs are fertilized in water supplemented with glutathione to prevent hardening of the chorion. At the one-cell stage, eggs are collected and divided up in 5 batches (10 eggs/batch) and the gapmers described in Example 1 are dissolved in physiological saline to prepare solutions of gapmers 1-4 at a concentration of 400 ng/μl. One batch of eggs is microinjected with a gapmer solution, i.e. batch 1/solution of gapmer 1, batch 2/solution of gapmer 2 and so on while batch 5 is injected with physiological saline only (same volume as gapmer solution). Microinjection is carried out using a World Precision instruments, PV820 pneumatic PicoPump, coupled with a Narishige MN-151 micromanipulator. After injection, the eggs are cultured in water containing glutathione at 8° C. in the dark for 24 hours. From each batch, 3 eggs are transferred into a test tube for RNA extraction, reverse transcription and PCR which is carried out as described in Example 1. The absence of or a weak PCR signal shows that the transfection is successful and that the gapmer is effective to target and destroy dnd-mRNA, i.e. knockdown of the dead end gene which is crucial for the migration and survival of PGCs. The remaining eggs are rinsed in physiological saline and transferred for further development into their natural habitat until reproductively sterile fish hatch therefrom.

### Example 3

### Transfection of Fertilized Salmo Salar Eggs by Immersion in an Aqueous Immersion Medium Comprising a Gapmer that Targets Dead End mRNA and a Transfection Agent

Salmo salar eggs are fertilized in an aqueous immersion medium (150 mM NaCl, 3.3 mM KCl, 20 mM Tris-HCl, pH 8.4) for 10 minutes before the fertilized eggs are collected and rinsed once in the same aqueous immersion medium. The eggs are placed into 10 test tubes (10 eggs/test tube) and immersed in an aqueous immersion medium (150 mM NaCl, 3.3 mM KCl, 20 mM Tris-HCl, pH 8.4) containing a pre-mix of the gapmer 1, 2, 3 or 4 and Oligofectamine™ (Thermofisher) following the manufacturer's instructions or only the transfection agent and no gapmer. The concentration of gapmer in the gapmer-containing immersion media is 8 nM. All test tubes are stored at 8° C. in the dark for 6 hours. From each test tube, 3 eggs are transferred into a fresh test tube for RNA extraction, reverse transcription and PCR which is carried out as described in Example 1. The absence of or a weak PCR signal shows that the transfection is successful and that the gapmer is effective to target and destroy dnd-mRNA, i.e. knockdown the of dead end gene which is crucial for the migration and survival of PGCs. The remaining eggs are rinsed in physiological saline and transferred for further development into their natural habitat until reproductively sterile fish hatch therefrom.

### Embodiments

1. A method for producing sterile egg-producing aquatic animals, said method comprising transfecting such eggs with a gapmer that is effective to render individuals produced therefrom sterile.
2. The method according to embodiment 1, wherein the gapmer has a sequence suitable for targeting RNA of genes that are essential for embryonic germ cell development.
3. The method according to embodiments 1 or 2, wherein the gapmer has a sequence 25 suitable for targeting dead end (dnd), nanos, vasa, piwi, gnrh or fsh receptor RNA.
4. The method according to embodiment 3, wherein the gapmer has a sequence suitable for targeting dnd RNA.
5. The method according to any of the preceding embodiments, wherein the gapmer comprises a gap that comprises a continuous stretch of RNase H recruiting nucleotides and wherein said gap is flanked by a 5′ wing region and a 3′ wing region, each of which comprise affinity-enhancing chemically modified nucleotides.
6. The method according to embodiment 5, wherein each nucleotide of the gap is a 2′ deoxynucleotide.
7. The method according to embodiment 6, wherein one of more of the 2′deoxynucleotides are substituted by a modified nucleotide that is DNA-like.
8. The method according to embodiment 6, wherein one or more of the 2′deoxynucleotides are substituted by a non-DNA-like nucleotide and wherein the gapmer nonetheless supports RNase H activation by virtue of the number or placement of the non-DNA-like nucleotides.
9. The method according to any of embodiments 5 to 8, wherein the 5′ wing region of the gapmer consists of 1 to 8 linked nucleotides.
10. The method according to any of embodiments 5 to 9, wherein the 3′ wing region of the gapmer consists of 1 to 8 linked nucleotides.
11. The method according to any of embodiments 5 to 10, wherein the 5′ wing region of the gapmer consists of the same number of linked nucleotides as the 3′ wing region.
12. The method according to any of embodiments 5 to 10, wherein the 5′ wing region of the gapmer consists of a different number of linked nucleotides than the 3′ wing region.
13. The method according to any of embodiments 5 to 12, wherein the 5′ wing region of the gapmer and/or the 3′ wing region of the gapmer comprise LNA and/or 2′alkylated RNA nucleotides.
14. The method according to embodiment 13, wherein the 5′ wing region of the gapmer and/or the 3′ wing region of the gapmer consist of LNA.
15. The method according to any of the preceding embodiments, wherein the gapmer consists of 8 to 36 nucleotides, e.g. 10 to 22 nucleotides, such as 12 to 18, 13 to 17 or 12 to 16 nucleotides, e.g. 12, 13, 14, 15 or 16 nucleotides or 14, 15, 16, 17, 18, 19 or 20 nucleotides.
16. The method according to any of embodiments 5 to 15, wherein the wing-gap-wing motif of the gapmer is 5-8-5, 5-6-5, 4-10-4, 4-8-4, 4-6-4, 3-12-3, 3-10-3, 3-8-3, 2-16-2, 2-14-2, 2-12-2, 2-10-2, 1-16-1, 1-14-1, 1-12-1, 1-10-1, 2-8-2, 1-8-1, 3-6-3 or 1-6-1.
17. The method according to any of the preceding embodiments, wherein the internucleoside linkages between the nucleotides in the gapmer are all phosphorothioate internucleoside linkages.
18. The method according to any of the preceding embodiments wherein the eggs are either unfertilized eggs or pre-water activated fertilized eggs or fertilized eggs.
19. The method according to any of the preceding embodiments, wherein the eggs are transfected by immersion in an aqueous immersion medium that comprises the gapmer.
20. The method according to embodiment 19, wherein the immersion medium further comprises other compounds, including compounds that assist or promote the transfection and/or compounds that are beneficial to the embryos/egg-producing aquatic animals hatched from the transfected eggs.
21. The method according to embodiment 20, wherein the immersion medium further comprises compounds selected from salts, buffering agents, chelating agents and amino acids, hormones, growth promoters, protective antigens, antibiotics, nutrients, ovarian fluid, serum and protease inhibitors.
22. The method according to any of embodiments 19 to 21, wherein the gapmer is taken up into the eggs by gymnosis.
23. The method according to embodiment 22, wherein the eggs are unfertilized eggs or 5 pre-water activated fertilized eggs.
24. The method according to embodiment 22, wherein the eggs are fertilized eggs.
25. The method according to any of embodiments 22 to 24, wherein the concentration of the gapmer in the immersion medium is in the range of 1 nM to 250 μM, e.g. 1 nM to 5 nM or 5 nM to 50 nM or 10 nM to 20 nM or 100 nM to 250 μM, e.g. 100 to 250 nM, 250 to 500 nM, 500 to 1000 nM, 1 μM to 50 μM, 50 μM to 100 μM, 100 μM to 150 μM, 150 μM to 200 μM or 200 μM to 250 μM, such as at least 5 nM, at least 10 nM, at least 20 nM, at least 50 nM, at least 100 nM, at least 250 nM, at least 500 nM, at least 1 μM, at least 50 μM, at least 100 μm, at least 150 μM, at least 200 μM.
26. The method according to any of embodiments 22 to 25, wherein the eggs are immersed in the aqueous immersion medium comprising the gapmer for 1 to 144 hours, e.g. 1 hour to 2 hours, 2 hours to 6 hours, 6 hours to 12 hours, 12 hours to 24 hours, 24 hours to 36 hours, 36 hours to 48 hours, 48 hours to 120 hours or 4 hours to 12 hours, 24 hours to 96 hours, or 36 to 72 hours, such as at least 2 hours, at least 4 hours, at least 6 hours, at least 8 hours, at least 12 hours, at least 24 hours, at least 36 hours, at least 48 hours, at least 60 hours, at least 72 hours, at least 84 hours, at least 96 hours, at least 108 hours, at least 120 hours or at least 132 hours.
27. The method according to any of embodiments 19 to 21, wherein the eggs are transfected by electroporation.
28. The method according to embodiment 27, wherein the eggs are immersed in the aqueous immersion medium comprising the gapmer for milliseconds to seconds.
29. The method according to any of embodiment 19 to 21, wherein the immersion medium comprises a transfection agent.
30. The method according to embodiment 29, wherein the transfection agent is a cationic agent, preferably a cationic lipid/cationic liposome, a cationic polymer, a cationic dendrimer or a cationic peptide.
31. The method according to embodiment 30, wherein the transfection agent comprises either 2,3-dioleyloxy-N-[2(sperminecarboxamido)ethyl]-N,N-dimethyl-1-propanaminium trifluoroacetate (DOSPA) or N-[1-(2,3-dioleyloxy)propyl]-n,n,n-trimethylammonium chloride (DOTMA) together with dioleoyl phosphatidylethanolamine (DOPE).
32. The method according to any of embodiments 29 to 31, wherein the concentration of the gapmer in the aqueous immersion medium is about 0.1 nM to 100 μM.
33. The method according to any of embodiments 29 to 32, wherein the eggs are fertilized eggs and the concentration of the gapmer in the aqueous immersion medium is about 1 nM to 100 μM, e.g. 1 nM to 100 nM, 100 nM to 1 μM, 1 μM to 20 μM, 20 μM to 80 μM, 20 μM to 60 μM or 20 μM to 40 μM, such as at least 1 nM, at least 10 nM, at least 50 nM, at least 100 nM, at least 1 μM, at least 10 μM, at least 20 μM, at least 40 μM, at least 60 μM or at least 80 μM.
34. The method according to any of embodiments 29 to 32, wherein the eggs are unfertilized eggs or pre-water activated fertilized eggs and the concentration of the gapmer in the aqueous immersion medium is about0.1 nm to 1 nM, 1 nM to 10 nM, 10 nM to 100 nM, 1 μM to 40 μM, e.g. 1 μM to 30 μM, 1 μM to 20 μM, 1 μM to 15 μM, 1 μM to 10 μM or 1 μM to 5 μM, such as at least 0.1 nM, at least 1 nM, at least 10 nM, at least 100 nM, at least 1 μM, at least 3 μM, at least 5 μM, at least 10 μM, at least 15 μM, at least 20 μM or at least 30 μM.
35. The method according to any of embodiments 29 to 34, wherein the eggs are immersed in the immersion medium comprising the gapmer and the transfection agent for 1 to 96 hours, e.g. 1 to 2 hours, 2 to 4 hours, 4 to 8 hours, 8 to 10 hours, 10 to 12 hours, 12 to 24 hours, 24 to 48 hours, 48 to 72 hours or 72 to 96 hours.
36. The method according to any of embodiments 19 to 21, wherein the eggs are transfected by microinjection.
37. The method according to embodiment 36, wherein the eggs are fertilized eggs at the one cell stage.
38. The method according to embodiments 36 or 37, wherein the gapmer is injected at a concentration of 5-5000 ng/μl, e.g. 50-500 ng/μl or 100-400 ng/μl.
39. The method according to any of the preceding embodiments, wherein the egg-producing aquatic animal is a fish, a crustacean or a mollusc.
40. The method according to embodiment 39, wherein the egg-producing aquatic animal is a fish.
41. The method according to embodiment 40, wherein said fish is a salmonid, moronid, cichlid, gadid, pangasid, ictalurid or cyprinid.
42. The method according to embodiment 40, wherein said fish is an Atlantic salmon, coho salmon, chinook salmon, chum salmon, sockeye salmon, pink salmon, masu salmon, rainbow trout, brook trout, brown trout, common grayling, Arctic grayling, Arctic char, bass, striped bass, white bass, striped-white bass hybrids, yellow bass, white perch, yellow perch European perch, bass-perch hybrid, Nile tilapia, blue tilapia, blue Nile tilapia hybrid, Mozambique tilapia, zebrafish seabream, porgy, cod, haddock, whiting, pollock or catfish.
43. The method according to embodiment 39, wherein the egg-producing aquatic animal is a crustacean.
44. The method according to embodiment 43, wherein said crustacean is a shrimp, a prawn, a lobster, a crayfish or a crab.
45. The method according to embodiment 39, wherein said egg-producing animal is a mollusc.
46. The method according to embodiment 45, wherein said mollusc is an oyster, mussel, scallop, geoduck, squid, abalone, octopus or cuttlefish.
47. A gapmer for use in a method for producing sterile egg-producing aquatic animals, wherein said method comprises transfecting such eggs with the gapmer, which is effective to render individuals, produced therefrom sterile.
48. The gapmer for use in the method according to embodiment 47, wherein the gapmer has a sequence suitable for targeting RNA of genes that are essential for embryonic germ cell development.
49. The gapmer for use in the method according to embodiments 47 or 48, wherein the gapmer has a sequence suitable for targeting dead end (dnd), nanos, vasa, piwi, gnrh or fsh receptor RNA.
50. The gapmer for use in the method according to embodiment 49, wherein the gapmer has a sequence suitable for targeting dnd RNA.
51. The gapmer for use in the method according to any of embodiments 47 to 50, wherein the gapmer comprises a gap that comprises a continuous stretch of RNase H recruiting nucleotides and wherein said gap is flanked by a 5′ wing region and a 3′ wing region, each of which comprise affinity-enhancing chemically modified nucleotides.
52. The gapmer for use in the method according to embodiment 51, wherein each nucleotide of the gap is a 2′-deoxynucleotide.
53. The gapmer for use in the method according to embodiment 52, wherein one of more of the 2′-deoxynucleotides are substituted by a modified nucleotide that is DNA-like.
54. The gapmer for use in the method according to embodiment 52, wherein one or more 5 of the 2′-deoxynucleotides are substituted by a non-DNA-like nucleotide and wherein the gapmer nonetheless supports RNase H activation by virtue of the number or placement of the non-DNA-like nucleotides.
55. The gapmer for use in the method according to embodiments 51 to 54, wherein the 5′ wing region of the gapmer consists of 1 to 8 linked nucleotides.
56. The gapmer for use in the method according to embodiments 51 to 55, wherein the 3′ wing region of the gapmer consists of 1 to 8 linked nucleotides.
57. The gapmer for use in the method according to embodiments 51 to 56, wherein the 5′ wing region of the gapmer consists of the same number of linked nucleotides as the 3′ wing region.
58. The gapmer for use in the method according to embodiments 51 to 56, wherein the 5′ wing region of the gapmer consists of a different number of linked nucleotides than the 3′ wing region.
59. The gapmer for use in the method according to embodiments 51 to 58, wherein the 5′ wing region of the gapmer and/or the 3′ wing region of the gapmer comprise LNA 25 and/or 2′-alkylated RNA nucleotides.
60. The gapmer for use in the method according to embodiment 59, wherein the 5′ wing region of the gapmer and/or the 3′ wing region of the gapmer consist of LNA.
61. The gapmer for use in the method according to embodiments 46 to 60, wherein the gapmer consists of 8 to 36 nucleotides, e.g. 10 to 22 nucleotides, such as 12 to 18, 13 to 17 or 12 to 16 nucleotides, e.g. 12, 13, 14, 15 or 16 nucleotides or 14, 15, 16, 17, 18, 19 or 20 nucleotides.
62. The gapmer for use in the method according to embodiments 51 to 61, wherein the 5 wing-gap-wing motif of the gapmer is 5-8-5, 5-6-5, 4-10-4, 4-8-4, 4-6-4, 3-12-3, 3-103, 3-8-3, 2-16-2, 2-14-2, 2-12-2, 2-10-2, 1-16-1, 1-14-1, 1-12-1, 1-10-1, 2-8-2, 1-8-1, 36-3 or 1-6-1.
63. The gapmer for use in the method according to embodiments 46 to 62, wherein the internucleoside linkages between the nucleotides in the gapmer are all phosphorothioate internucleoside linkages.
64. The gapmer for use in the method according to embodiments 46 to 63 wherein the eggs are either unfertilized eggs or pre-water activated fertilized eggs or fertilized eggs.
65. The gapmer for use in the method according to embodiments 46 to 64, wherein the eggs are transfected by immersion in an aqueous immersion medium that comprises the gapmer.
66. The gapmer for use in the method according to embodiment 65, wherein the immersion medium further comprises other compounds, including compounds that assist or promote the transfection and/or compounds that are beneficial to the embryos/egg-producing aquatic animals hatched from the transfected eggs.
67. The gapmer for use in the method according to embodiment 66, wherein the immersion medium further comprises compounds selected from salts, buffering agents, chelating agents and amino acids, hormones, growth promoters, protective antigens, antibiotics, nutrients, ovarian fluid, serum and protease inhibitors.
68. The gapmer for use in the method according to embodiments 65 to 67, wherein the gapmer is taken up into the eggs by gymnosis.
69. The gapmer for use in the method according to embodiment 68, wherein the eggs are unfertilized eggs or pre-water activated fertilized eggs.
70. The gapmer for use in the method according to embodiment 68, wherein the eggs are fertilized eggs.
71. The gapmer for use in the method according to embodiments 68 to 70, wherein the concentration of the gapmer in the immersion medium is in the range of 1 nM to 250 μM, e.g. 1 nM to 5 nM or 5 nM to 50 nM or 10 nM to 20 nM or 100 nM to 250 μM, e.g. 100 to 250 nM, 250 to 500 nM, 500 to 1000 nM, 1 μM to 50 μM, 50 μM to 100 μM, 100 μM to 150 μM, 150 μM to 200 μM or 200 μM to 250 μM, such as at least 5 nM, at least 10 nM, at least 20 nM, at least 50 nM, at least 100 nM, at least 250 nM, at least 500 nM, at least 1 μM, at least 50 μM, at least 100 μm, at least 150 μM, at least 200 μM.
72. The gapmer for use in the method according to embodiments 68 to 71, wherein the eggs are immersed in the aqueous immersion medium comprising the gapmer for 1 to 144 hours, e.g. 1 hour to 2 hours, 2 hours to 6 hours, 6 hours to 12 hours, 12 hours to 24 hours, 24 hours to 36 hours, 36 hours to 48 hours, 48 hours to 120 hours or 4 hours to 12 hours, 24 hours to 96 hours, or 36 to 72 hours, such as at least 2 hours, at least 4 hours, at least 6 hours, at least 8 hours, at least 12 hours, at least 24 hours, at least 36 hours, at least 48 hours, at least 60 hours, at least 72 hours, at least 84 hours, at least 96 hours, at least 108 hours, at least 120 hours or at least 132 hours.
73. The gapmer for use in the method according to embodiments 65 to 67, wherein the eggs are transfected by electroporation.
74. The gapmer for use in the method according to embodiment 73, wherein the eggs are immersed in the aqueous immersion medium comprising the gapmer for milliseconds to seconds.
75. The gapmer for use in the method according to embodiments 65 to 67, wherein the immersion medium comprises a transfection agent.
76. The method according to embodiment 75, wherein the transfection agent is a cationic agent, preferably a cationic lipid/cationic liposome, a cationic polymer, a cationic dendrimer or a cationic peptide.
77. The gapmer for use in the method according to embodiment 76, wherein the transfection agent comprises either 2,3-dioleyloxy-N-[2(sperminecarboxamido)ethyl]-N,N-dimethyl-1-propanaminium trifluoroacetate (DOSPA) or N-[1-(2,3-dioleyloxy)propyl]-n,n,n-trimethylammonium chloride (DOTMA) together with dioleoyl phosphatidylethanolamine (DOPE).
78. The gapmer for use in the method according to embodiments 75 to 77, wherein the concentration of the gapmer in the aqueous immersion medium is about 0.1 nM to 100 μM.
79. The gapmer for use in the method according to embodiments 75 to 78, wherein the eggs are fertilized eggs and the concentration of the gapmer in the aqueous immersion medium is about 1 nM to 100 μM, e.g. 1 nM to 100 nM, 100 nM to 1 μM, 1 μM to 20 μM, 20 μM to 80 μM, 20 μM to 60 μM or 20 μM to 40 μM, such as at least 1 nM, at least 10 nM, at least 50 nM, at least 100 nM, at least 1 μM, at least 10 μM, at least 20 μM, at least 40 μM, at least 60 μM or at least 80 μM.
80. The gapmer for use in the method according to embodiments 75 to 78, wherein the eggs are unfertilized eggs or pre-water activated fertilized eggs and the concentration of the gapmer in the aqueous immersion medium is about0.1 nm to 1 nM, 1 nM to 10 nM, 10 nM to 100 nM, 1 μM to 40 μM, e.g. 1 μM to 30 μM, 1 μM to 20 μM, 1 μM to 15 μM, 1 μM to 10 μM or 1 μM to 5 μM, such as at least 0.1 nM, at least 1 nM, at least 10 nM, at least 100 nM, at least 1 μM, at least 3 μM, at least 5 μM, at least 10 μM, at least 15 μM, at least 20 μM or at least 30 μM.
81. The gapmer for use in the method according to embodiments 75 to 80, wherein the eggs are immersed in the immersion medium comprising the gapmer and the transfection agent for 1 to 96 hours, e.g. 1 to 2 hours, 2 to 4 hours, 4 to 8 hours, 8 to 10 hours, 10 to 12 hours, 12 to 24 hours, 24 to 48 hours, 48 to 72 hours or 72 to 96 hours.
82. The gapmer for use in the method according to embodiments 65 to 67, wherein the 5 eggs are transfected by microinjection.
83. The gapmer for use in the method according to embodiment 82, wherein the eggs are fertilized eggs at the one cell stage.
84. The gapmer for use in the method according to embodiments 82 or 83, wherein the gapmer is injected at a concentration of 5-5000 ng/μl, e.g. 50-500 ng/μl or 100-400 ng/μl.
85. The gapmer for use in the method according to embodiments 46 to 84, wherein the egg-producing aquatic animal is a fish, a crustacean or a mollusc.
86. The gapmer for use in the method according to embodiment 85, wherein the egg-producing aquatic animal is a fish.
87. The gapmer for use in the method according to embodiment 86, wherein said fish is a salmonid, moronid, cichlid, gadid, pangasid, ictalurid or cyprinid.
88. The gapmer for use in the method according to embodiment 86, wherein said fish is an Atlantic salmon, coho salmon, chinook salmon, chum salmon, sockeye salmon, pink salmon, masu salmon, rainbow trout, brook trout, brown trout, common grayling, Arctic grayling, Arctic char, bass, striped bass, white bass, striped-white bass hybrids, yellow bass, white perch, yellow perch European perch, bass-perch hybrid, Nile tilapia, blue tilapia, blue-Nile tilapia hybrid, Mozambique tilapia, zebrafish seabream, porgy, cod, haddock, whiting, pollock or catfish.
89. The gapmer for use in the method according to embodiment 85, wherein the egg-producing aquatic animal is a crustacean.
90. The gapmer for use in the method according to embodiment 89, wherein said crustacean is a shrimp, a prawn, a lobster, a crayfish or a crab.
91. The gapmer for use in the method according to embodiment 85, wherein said egg 5 producing animal is a mollusc.
92. The gapmer for use in the method according to embodiment 91, wherein said mollusc is an oyster, mussel, scallop, geoduck, squid, abalone, octopus or cuttlefish.
93. A gapmer which is effective to transfect eggs from egg-producing aquatic animals and render individuals produced therefrom sterile.
94. The gapmer according to embodiment 93 which suppresses expression of a protein that is essential for embryonic germ cell development in said egg-producing aquatic animals.
95. The gapmer according to embodiments 93 or 94, wherein the gapmer has a sequence suitable for targeting RNA of genes that are essential for embryonic germ cell development in the egg-producing aquatic animals.
96. The gapmer according to embodiments 93 to 95, wherein the gapmer has a sequence suitable for targeting dead end (dnd), nanos, vasa, piwi, gnrh or fsh receptor RNA.
97. The gapmer according to embodiment 96, wherein the gapmer has a sequence 25 suitable for targeting dnd RNA.
98. The gapmer according to any of embodiments 96 to 97, wherein said RNA is an RNA from a fish, a crustacean or a mollusc.
99. The gapmer according to embodiment 98, wherein said RNA is RNA from a fish.
100. The gapmer according to embodiment 99, wherein said fish is a salmonid, moronid, cichlid, gadid, pangasid, ictalurid or cyprinid.
101. The gapmer according to embodiment 99, wherein said fish is an Atlantic salmon, coho salmon, chinook salmon, chum salmon, sockeye salmon, pink salmon, masu salmon, rainbow trout, brook trout, brown trout, common grayling, Arctic grayling, Arctic char, bass, striped bass, white bass, striped-white bass hybrids, yellow bass, white perch, yellow perch European perch, bass-perch hybrid, Nile tilapia, blue tilapia, blueNile tilapia hybrid, Mozambique tilapia, zebrafish seabream, porgy, cod, haddock, whiting, pollock or catfish.
102. The gapmer according to embodiment 98, wherein said RNA is RNA from a crustacean.
103. The gapmer according to embodiment 102, wherein said crustacean is a shrimp, a prawn, a lobster, a crayfish or a crab.
104. The gapmer according to embodiment 98, wherein said RNA is RNA from a mollusc.
105. The gapmer according to embodiment 104, wherein said mollusc is an oyster, mussel, scallop, geoduck, squid, abalone, octopus or cuttlefish.
106. The gapmer according to any of embodiments 93 to 105, wherein the gapmer comprises a gap that comprises a continuous stretch of RNase H recruiting nucleotides and wherein said gap is flanked by a 5′ wing region and a 3′ wing region, each of which comprise affinity-enhancing chemically modified nucleotides.
107. The gapmer according to embodiment 106, wherein each nucleotide of the gap is a 2′-deoxynucleotide.
108. The gapmer according to embodiment 107, wherein one of more of the 2′deoxynucleotides are substituted by a modified nucleotide that is DNA-like.
109. The gapmer according to embodiment 107, wherein one or more of the 2′deoxynucleotides are substituted by a non-DNA-like nucleotide and wherein the gapmer nonetheless supports RNase H activation by virtue of the number or placement of the non-DNA-like nucleotides.
110. The gapmer according to any of embodiments 106 to 109, wherein the 5′ wing region of the gapmer consists of 1 to 8 linked nucleotides.
111. The gapmer according to any of embodiments 106 to 110, wherein the 3′ wing region of the gapmer consists of 1 to 8 linked nucleotides.
112. The gapmer according to any of embodiments 106 to 111, wherein the 5′ wing region of the gapmer consists of the same number of linked nucleotides as the 3′ wing region.
113. The gapmer according to any of embodiments 106 to 111, wherein the 5′ wing region of the gapmer consists of a different number of linked nucleotides than the 3′ wing region.
114. The gapmer according to any of embodiments 106 to 113, wherein the 5′ wing region of the gapmer and/or the 3′ wing region of the gapmer comprise LNA and/or 2′-alkylated RNA nucleotides.
115. The gapmer according to embodiment 114, wherein the 5′ wing region of the gapmer and/or the 3′ wing region of the gapmer consist of LNA.
116. The gapmer according to any of embodiments 106 to 115, wherein the gapmer consists of 8 to 36 nucleotides, e.g. 10 to 22 nucleotides, such as 12 to 18, 13 to 17 or 12 to 16 nucleotides, e.g. 12, 13, 14, 15 or 16 nucleotides or 14, 15, 16, 17, 18, 19 or 20 nucleotides.
117. The gapmer according to any of embodiments 106 to 116, wherein the wing-gap-wing motif of the gapmer is 5-8-5, 5-6-5, 4-10-4, 4-8-4, 4-6-4, 3-12-3, 3-10-3, 3-8-3, 2-16-2, 2-14-2, 2-12-2, 2-10-2, 1-16-1, 1-14-1, 1-12-1, 1-10-1, 2-8-2, 1-8-1, 3-6-3 or 1-6-1.
118. The gapmer according to any of embodiments 106 to 117, wherein the internucleoside linkages between the nucleotides in the gapmer are all phosphorothioate internucleoside linkages.
119. The gapmer according to embodiment 97, wherein said dnd RNA is that of salmonids.
120. The gapmer according to embodiment 119, wherein said salmonid is Atlantic salmon, coho salmon, chinook salmon, chum salmon, sockeye salmon, pink salmon, masu salmon, rainbow trout, brook trout, brown trout, common grayling, Arctic grayling or Arctic char.
121. The gapmer according to embodiment 120, wherein said salmonid is Atlantic salmon.
122. The gapmer according to embodiment 121, wherein said gapmer has a sequence selected from SED ID NO: 1, SED ID NO: 2 or SED ID NO: 3 or a variant thereof.
123. The gapmer according to embodiment 122, wherein said gapmer has a sequence selected from SED ID NO: 1, SED ID NO: 2 or SED ID NO: 3.
124. A composition comprising the gapmer according to any of embodiments 93 to 123 and a pharmaceutically acceptable carrier.
125. A composition according to embodiment 124, wherein said composition comprises two or more gapmers.
126. The composition according to embodiment 125, wherein said composition comprises the gapmer of SED ID NO: 1 and the gapmer of SED ID NO: 2 or variants thereof or the gapmer of SED ID NO: 1 and the gapmer of SED ID NO: 3 or variants thereof or the gapmer of SED ID NO: 2 and the gapmer SED ID NO: 3 or variants thereof or the gapmer of SED ID NO: 1 and the gapmer of SED ID NO: 2 and the gapmer of SED ID NO: 3 or variants thereof.
127. The composition according to embodiment 126, wherein said composition comprises the gapmer of SED ID NO: 1 and the gapmer of SED ID NO: 2 or the gapmer of SED ID NO: 1 and the gapmer of SED ID NO: 3 or the gapmer of SED ID NO: 2 and the gapmer SED ID NO: 3 or the gapmer of SED ID NO: 1 and the gapmer of SED ID NO: 2 and the gapmer of SED ID NO: 3.
128. The composition according to any of embodiments 124 to 127, wherein the pharmaceutically acceptable carrier is water or an aqueous buffer.
129. A method for producing sterile fish, said method comprising transfecting such fish eggs with a gapmer that has a sequence suitable for targeting dnd RNA by immersing the eggs in an aqueous immersion medium comprising said gapmer.
130. The method according to embodiment 129, wherein said immersion medium comprises the gapmer at a concentration of 1 nM to 100 nM, preferably 5 nM to 50 nM.
131. The method according to embodiments 129 or 130, wherein the immersion medium consists of an aqueous buffer and the gapmer.
132. The method according to embodiments 129 to 131, wherein the eggs are immersed in the aqueous immersion medium for 1 to 48 hours, preferably 2 to 24 hours.
133. The method according to embodiments 129 to 132, wherein the eggs are salmonid eggs.
134. The method according to embodiments 129 to 133, wherein the eggs are from Atlantic salmon.
135. The method according to embodiment 134, wherein the gapmer has a sequence selected from SED ID NO: 1, SED ID NO: 2 and SED ID NO: 3 or a variant thereof.
136. The method according to embodiment 135, wherein the gapmer has a sequence selected from SED ID NO: 1, SED ID NO: 2 and SED ID NO: 3

While the disclosure has been has been set out herein in reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the invention as hereinafter claimed is intended to be broadly construed and interpreted, as including all such variations, modifications and alternative embodiments, within its spirit and scope.

1-31. (canceled)

32. A method for producing sterile egg-producing aquatic animals, said method comprising transfecting such eggs with a gapmer that is effective to render individuals produced therefrom sterile, wherein the gapmer has a sequence suitable for targeting RNA of genes that are essential for embryonic germ cell development and wherein the eggs are transfected by immersion in an aqueous immersion medium that comprises the gapmer.

33. The method according to claim 32, wherein the gapmer has a sequence suitable for targeting dead end (dnd), nanos, vasa, piwi, gnrh or fsh receptor RNA.

34. The method according to claim 32 wherein the gapmer has a sequence suitable for targeting dnd RNA.

35. The method according to claim 32, wherein the gapmer comprises a gap that comprises a continuous stretch of RNase H recruiting nucleotides and wherein said gap is flanked by a 5′ wing region and a 3′ wing region, each of which comprise affinity-enhancing chemically modified nucleotides.

36. The method according to claim 32, wherein the gapmer consists of 8 to 36 nucleotides.

37. The method according to claim 32, wherein the gapmer consists of 10 to 22 nucleotides.

38. The method according to claim 32, wherein the gapmer consists of 14 to 20 nucleotides.

39. The method according to claim 32, wherein the gapmer is taken up into the eggs by gymnosis.

40. The method according to claim 32, wherein the eggs are transfected by electroporation.

41. The method according to claim 32, wherein the immersion medium comprises a transfection agent.

42. The method according to claim 41, wherein the transfection agent is a cationic agent.

43. The method according to claim 41, wherein the transfection agent is selected from the group consisting of cationic lipid, cationic liposome, cationic polymer, cationic dendrimer and cationic peptide.

44. The method according to claim 32, wherein the egg-producing aquatic animal is a fish.

45. The method according to claim 44, wherein the fish is selected from the group consisting of salmonid, moronid, cichlid, gadid, pangasid, ictalurid and cyprinid.

46. The method according to claim 32, wherein the egg-producing aquatic animal is an Atlantic salmon and wherein the gapmer has sequence selected from the group consisting of SED ID NO: 1, SED ID NO: 2 and SED ID NO: 3 or a variant thereof.

47. A gapmer having a sequence suitable for targeting dnd RNA of salmonids, which is effective to transfect eggs from salmonids and render individuals produced therefrom sterile.

48. The gapmer according to claim 47, wherein the gapmer has a sequence selected from the group consisting of SED ID NO: 1, SED ID NO: 2 and SED ID NO: 3 or a variant thereof.

49. The gapmer according to claim 47, wherein the gapmer has a sequence selected from the group consisting of SED ID NO: 1, SED ID NO: 2 and SED ID NO: 3.

50. A composition comprising a gapmer having a sequence suitable for targeting dnd RNA of salmonids, which is effective to transfect eggs from salmonids and render individuals produced therefrom sterile and a pharmaceutically acceptable carrier.

51. The composition according to claim 50, wherein the gapmer has a sequence selected from the group consisting of SED ID NO: 1, SED ID NO: 2 and SED ID NO: 3 or a variant thereof.

52. The composition according to claim 50, wherein the gapmer has a sequence selected from the group consisting of SED ID NO: 1, SED ID NO: 2 and SED ID NO: 3.

53. The composition according to claim 50, wherein the pharmaceutically acceptable carrier is water or an aqueous buffer.


SEQUENCE LISTING (6 sequences)

SEQ ID NO: 1 - DNA, 16 bp, Salmo salar
ttgaacgctcctccat

SEQ ID NO: 2 - DNA, 16 bp, Salmo salar
ggagcagcgaggaggt

SEQ ID NO: 3 - DNA, 16 bp, Salmo salar
gcaaaacatttaagta

SEQ ID NO: 4 - DNA, 16 bp, Salmo salar
gctcccttcaatccaa

SEQ ID NO: 5 - DNA, 21 bp, Salmo salar
gagcgttcaagtcaggtgttg

SEQ ID NO: 6 - DNA, 20 bp, Salmo salar
cagagctgacgtttctccgt